(19) United States
(12) Patent Application Publication
(10) Pub. No.: US 2006/0166209 A1
(43) Pub. Date: Jul. 27, 2006

(54) Growth hormone variations in humans and its uses

(76) Inventors: David Neil Cooper, Cardiff (GB); Ann Marie Procter, Cardiff (GB); John Gregory, Cardiff (GB); David Stuart Millar, Cardiff (GB); Mark Lewis, Cardiff (GB); Angeles Ulied, Barcelona (ES)

Correspondence Address: KING & SCHICKLI, PLLC, 247 NORTH BROADWAY, LEXINGTON, KY, 40507, US

(21) Appl. No.: 10/535,005

(22) PCT Filed: Nov. 4, 2003
(86) PCT No.: PCT/GB03/04775

§ 371 Date: Nov. 18, 2005

(30) Foreign Application Priority Data
Nov. 12, 2002 (GB) 0226441.4
Apr. 10, 2003 (GB) 0308242.7
Nov. 12, 2002 (GB) PCTGB0205112

(51) Int. Cl. C12Q 1/68

(52) U.S. Cl. 435/6

(57) ABSTRACT

The present invention relates to a naturally-occurring growth hormone mutation; to a method for detecting it and its use in screening patients for growth hormone dysfunction or for producing variant proteins suitable for treating such irregularities.

The present invention relates to a naturally-occurring growth hormone mutation; and its use in screening patients for growth hormone irregularities or for producing variant therapies and therapeutics suitable for treating such irregularities.

That human stature was influenced by inherited factors was understood more than a century ago. Although familial short stature, with its normally recessive mode of inheritance, was recognised as early as 1912, it was a further quarter century before such families came to be properly documented in the scientific literature. The recognition that recessively inherited short stature was commonly associated with isolated growth hormone (GH) deficiency only came in 1966.

Short stature associated with GH deficiency has been estimated to occur with an incidence of between 1/4000 and 1/10000 live births. Most of these cases are both sporadic and idiopathic, but between 5 and 30% have an affected first-degree relative consistent with a genetic aetiology for the condition. Confirmation of the genetic aetiology of GH deficiency came from the molecular genetic analysis of familial short stature and the early demonstration of mutational lesions in the pituitary-expressed growth hormone. (GH1) genes of affected individuals. Familial short stature may also be caused by mutation in a number of other genes (eg POU1F1, PROP1 and GHRHR) and it is important to distinguish these different forms of the condition.

Growth hormone (GH) is a multifunctional hormone that promotes post-natal growth of skeletal and soft tissues through a variety of effects. Controversy remains as to the relative contribution of direct and indirect actions of GH. On one hand, the direct effects of GH have been demonstrated in a variety of tissues and organs, and GH receptors have been documented in a number of cell types. On the other hand, a substantial amount of data indicates that a major portion of the effects of GH are mediated through the actions of GH-dependent insulin-like growth factor I (IGF-I). IGF-1 is produced in many tissues, primarily the liver, and acts through its own receptor to enhance the proliferation and maturation of many tissues, including bone, cartilage, and skeletal muscle. In addition to promoting growth of tissues, GH has also been shown to exert a variety of other biological effects, including lactogenic, diabetogenic, lipolytic and protein anabolic effects, as well as sodium and water retention.

Adequate amounts of GH are needed throughout childhood to maintain normal growth. Newborns with GH deficiency are usually of normal length and weight. Some may have a micropenis or fasting hypoglycemia in conjunction with low linear postnatal growth, which becomes progressively retarded with age. In those with isolated growth hormone deficiency (IGHD), skeletal maturation is usually delayed in association with their height retardation. Truncal obesity, facial appearance younger than expected for their chronological age and delayed secondary dentition are often present. Skin changes similar to those seen in premature ageing may be seen in affected adults.

Familial IGHD comprises several different disorders with characteristic modes of inheritance. Those forms of IGHD known to be associated with defects at the GH1 gene locus are shown in Table 1 together with the different types of underlying lesion so far detected.

TABLE 1
Classification of inherited disorders involving the GH1 gene

| | Mode of | Types of gene | GH | |
|---|---|---|---|---|
| Disorder | inheritance | lesion responsible | protein | Deficiency state |
| IGHD IA | Autosomal | Gross deletions, | Absent | Severe short stature. |
| | recessive | micro-deletions, | | Anti-GH antibodies often |
| | | nonsense mutations | | produced upon GH |
| | | | | treatment, resulting in |
| | | | | poor response thereto. |
| IGHD IB | Autosomal | Splice site | Deficient | Short stature. Patients |
| | recessive | mutations | | usually respond well to |
| | | | | exogenous GH. |
| IGHD II | Autosomal | Splice site and | Deficient | Short stature. Patients |
| | dominant | intronic mutations, | | usually respond well to |
| | | missense mutations | | exogenous GH. |

The characterisation of these lesions has helped to provide explanations for the differences in clinical severity, mode of inheritance and propensity to antibody formation in response to exogenously administered GH, between these forms of IGHD. Most cases are sporadic and are assumed to arise from cerebral defects that include cerebral oedema, chromosomal anomalies, histiocytosis, infections, radiation, septo-optic dysplasia, trauma, or tumours affecting the hypothalamus or pituitary. Magnetic resonance imaging examinations detect hypothalamic or pituitary anomalies in about 12% of patients who have IGHD.

Although short stature, delayed ‘height velocity’ or growth velocity, and delayed skeletal maturation are all seen with GH deficiency, none of these is specific for this disorder; other systemic diseases may result in such symptoms. Throughout this specification, ‘height velocity’ and growth velocity are both to be construed as meaning the rate of change of the subject's or patient's height, such as is measured in centimetres per year.

Stimulation tests to demonstrate GH deficiency use L-Dopa, insulin-induced hypoglycaemia, arginine, insulin-arginine, clonidine, glucagon or propranolol. Inadequate GH peak responses (usually <7-10 ng/mL) differ from test to test. Testing for concomitant deficiencies of LH, FSH, TSH and ACTH should be performed to determine the extent of pituitary dysfunction and to plan optimal treatment.

Recombinant-derived GH is available worldwide and is administered by subcutaneous injection. To obtain an optimal outcome, children with IGHD are usually started on replacement therapy as soon as their diagnosis is established. The initial dosage of recombinant GH is based on body weight or surface area, but the exact amount used and the frequency of administration may vary between different protocols. The dosage increases with increasing body weight to a maximum during puberty. Thereafter, GH treatment should be temporarily discontinued while the individual's GH secretory capacity is re-evaluated. Those with confirmed GH deficiency receive a lower dose of exogenous GH during adult life.

Conditions that are treated with GH include (i) those in which it has proven efficacy and (ii) a variety of others in which its use has been reported but not accepted as standard practice. Disorders in which GH treatment has proven efficacy include GH deficiency, either isolated or in association with combined pituitary hormone deficiency (CPHD) and Turner syndrome. The clinical responses of individuals with the first two disorders to GH replacement therapy varies depending on: (i) the severity of the GH deficiency and its adverse effects on growth, the age at which treatment is begun, weight at birth, current weight and dose of GH; and (ii) recognition and response to treatment of associated deficiencies such as thyroid hormone deficiency; and (iii) whether treatment is complicated by the development of anti-GH antibodies. The outcome, of treatment for individuals with Turner syndrome varies with the severity of their short stature, their chromosomal complement, and the age at which treatment was begun.

Additional disorders in which the use of GH has been reported include treatment of certain skeletal dysplasias such as achondroplasia, Prader-Willi syndrome, growth suppression secondary to exogenous steroids or in association with chronic inflammatory diseases such as rheumatoid arthritis, in chronic renal failure, extreme idiopathic short stature, Russell-Silver syndrome, and intrauterine growth retardation.

The characterisation of familial IGHD at the molecular genetic level is important for several reasons. The identity of the locus involved will indicate not only the likely severity of growth retardation but, more importantly, the appropriateness or otherwise of the various therapeutic regimens now available. Further, detection of the underlying gene lesions serves to confirm the genetic aetiology of the condition. It may also have prognostic value in predicting (i) the severity of growth retardation and (ii) the likelihood of anti-GH antibody formation subsequent to GH treatment. In some instances, knowledge of the pathological lesion(s) can also help to explain an unusual mode of inheritance of the disorder and is therefore essential for the counselling of affected families. Finally, the characterisation of the mutational lesions responsible for cases of IGHD manifesting a dysfunctional (as opposed to a non-functional) GH molecule could yield new insights into GH structure and function.

At the cellular level, a single GH molecule binds two GH receptor molecules (GHR) causing them to dimerise. Dimerisation of the two GH-bound GHR molecules is believed to be necessary for signal transduction, which is associated with the tyrosine kinase JAK2. The intracellular tyrosine kinase, JAK2, is associated with the cytoplasmic tail of the GHR. Following GH binding, two JAK2 molecules are brought into close proximity resulting in cross-phosphorylation both of each other and of tyrosine residues on the cytoplasmic tail of the GHR. These phosphotyrosines act as docking points for cell signalling intermediates such as STAT 5. STAT 5 binding to the phosphorylated receptor tail then brings it into close proximity to JAK2 resulting in its own phosphorylation by JAK 2. Phospho-STAT 5 dimerizes and translocates to the nucleus where it transactivates GH-responsive genes leading to the observed biological effects of GH. Until recently it had been assumed that GH signalling was mediated primarily by the JAK/STAT pathway. However, it is now known that GH can also activate the phosphatidylinositol 3′-kinase (PI3K) and p42/44 mitogen activated protein kinase (MAPK) pathways. Activation of STAT 5 and the PI3K pathway can induce hepatic IGF-1 production but the MAPK pathway does not appear to do so.

Activation of JAK 2 and MAPK are dependent upon different regions of the cytoplasmic domain of the GHR from those involved in STAT 5 activation. STAT 5 activation requires JAK 2-mediated phosphorylation of tyrosine residues 534, 566 and 627, located towards the C-terminal end of the cytoplasmic domain of the GHR that are not required for GH-induced MAPK activation [Hansen et al, J Biol Chem 271 12669-12673 (1996)]. By contrast, activation of JAK 2 and the MAPK pathway is dependent upon a 46-amino acid stretch containing a proline-rich (box 1) domain located adjacent to the cell membrane [Sotiropoulos et al., Endocrinology 135 1292-1298 (1994)]. Activation of MAPK following GHR activation appears to be complex, involving multiple mechanisms. One of these mechanisms is mediated by JAK 2-dependent activation of the Shc-Grb2-Sos-Ras pathway [VanderKuur et al, Biol Chem 270 7587-7593 (1995); VanderKuur et al., Endocrinology 138 4301-4307 (1997)] possibly involving multiple docking proteins such as IRS-1 [Liang et al., Endocrinology 141 3328-3336 (2000)], Gab-1 [Kim et al, Endocrinology 143 4856-4867 (2000)] and the EGF receptor [Yamauchi et al., Nature 390 91-96 (1997)]. An alternative JAK 2-independent mechanism of MAPK activation via Src-dependent activation of Ral and phospholipase D has recently been reported [Zhu et al., J Biol Chem 277 45592-45603 (2002)]. Full MAPK activation by GH requires activation of both JAK 2 and Src, although Src activation alone is sufficient for partial MAPK activation [Zhu et al, J Biol Chem 277 45592-45603 (2002)].

It has been suggested that the diverse effects of GH may be mediated by a single type of GHR molecule that can possess different cytoplasmic domains or phosphorylation sites in different tissues. When activated by JAK2, these differing cytoplasmic domains can lead to distinct phosphorylation pathways, one for growth effects and others for various metabolic effects.

GH is a 22 kDa protein secreted by the somatotroph cells of the anterior pituitary. X-ray crystallographic studies have shown GH to comprise a core of two pairs of parallel alpha helices arranged in an up-up-down-down fashion. This structure is stabilised by two intra-molecular disulphide linkages (Cys53-Cys165 and Cys182-Cys 189). Two growth hormone receptor (GHR) molecules bind to two structurally distinct sites on the GH molecule, a process which proceeds sequentially by GHR binding first at site 1 and then at site 2. The binding of GHR to GH potentiates dimerisation of the GHR molecules.

Scanning mutagenesis studies of the GH molecule have yielded a picture of the binding interactions between GH and its receptor whilst site-directed mutagenesis has been used to probe the function of specific residues. Thus, substitution of Gly120 (in the third alpha helix of human GH) by Arg results in the loss of GHR binding to site 2 thereby blocking GHR dimerisation. Similarly, residue Phe44 of the human GH protein is important for binding the prolactin receptor. Finally, residues Asp115, Gly119, Ala122 and Leu123 have been shown to be critical for the growth enhancing potential of the murine GH molecule.

Interaction of the dimerised GHR with the intracellular tyrosine protein kinase JAK2 leads to tyrosine phosphorylation of downstream signal transduction molecules, stimulation of mitogen-activated protein (MAP) kinases and induction of signal transducers and activators of transcription (STAT proteins). In this way, GH is able to influence the expression of multiple genes through a number of different signalling pathways.

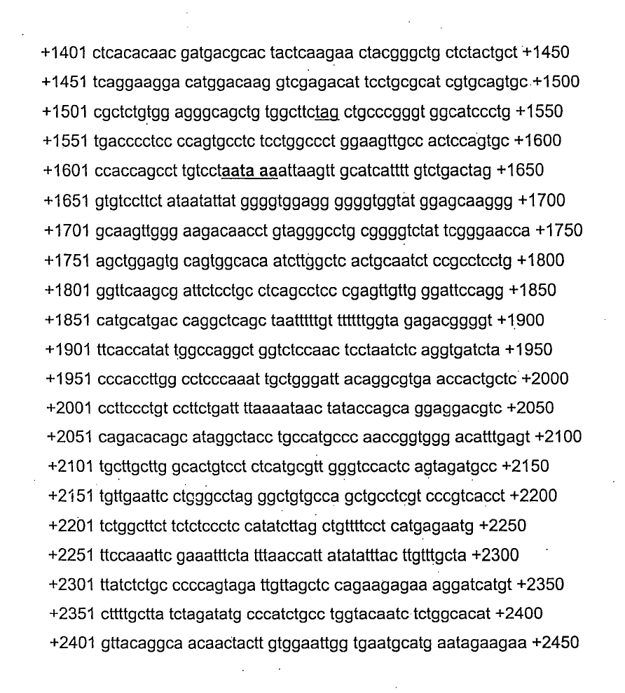

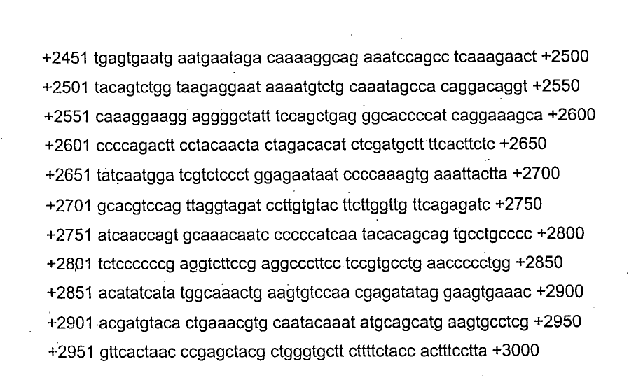

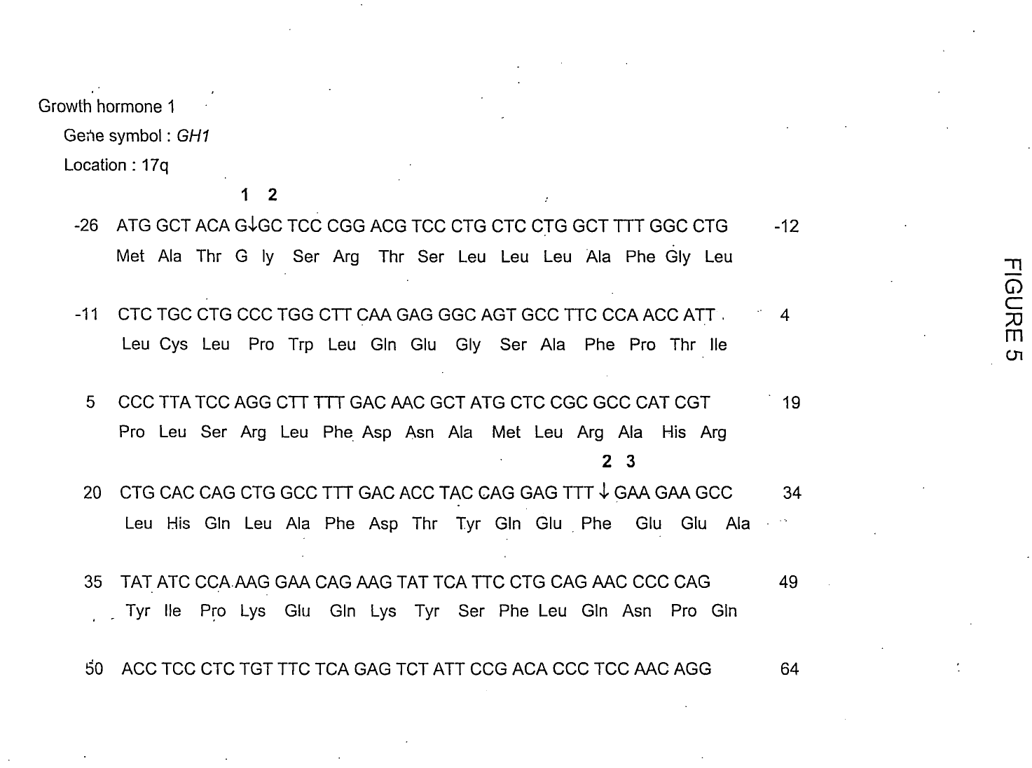

Several different GH isoforms are generated from expression of the GH1 gene (GH1 reference sequence is shown in FIG. 4). In 9% of GH1 transcripts, exon 2 is spliced to an alternative acceptor splice site 45 bp into exon 3, thereby deleting amino acid residues 32 to 46 and generating a 20 kDa isoform instead of the normal 22 kDa protein. This 20 kDa isoform appears to be capable of stimulating growth and differentiation. The factors involved in determining alternative acceptor splice site selection are not yet characterised but are clearly of a complex nature. A 17.5 kDa isoform, resulting from the absence of codons 32 to 71 encoded by exon 3, has also been detected in trace amounts in pituitary tumour tissue. Splicing products lacking either exons 3 and 4 or exons 2, 3 and 4 have been reported in pituitary tissue but these appear to encode inactive protein products. A 24 kDa glycosylated variant of GH has also been described. The amino acid sequence of the major 22 kDa isoform is presented in FIG. 5, which shows the nucleotide sequence of the GH1 gene coding region and amino acid sequence of the protein including the 26 amino acid leader peptide. Lateral numbers refer to amino acid residue numbering. Numbers in bold flanking vertical arrows specify the exon boundaries. The termination codon is marked with an asterisk.

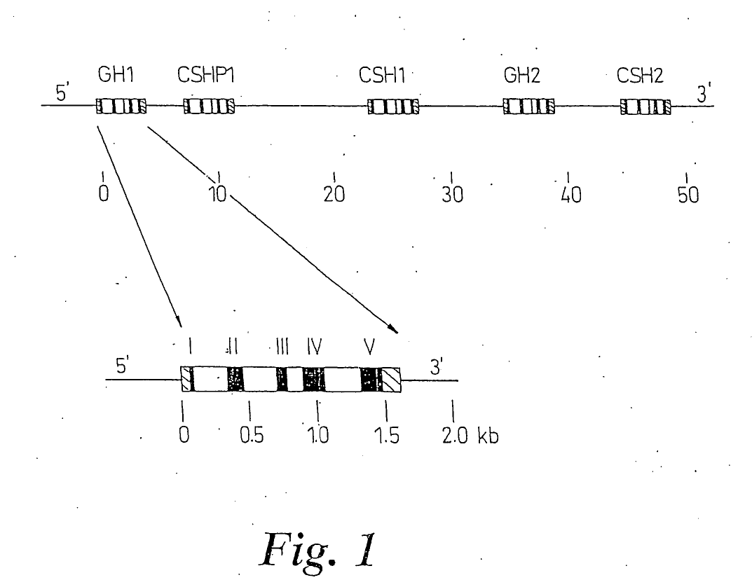

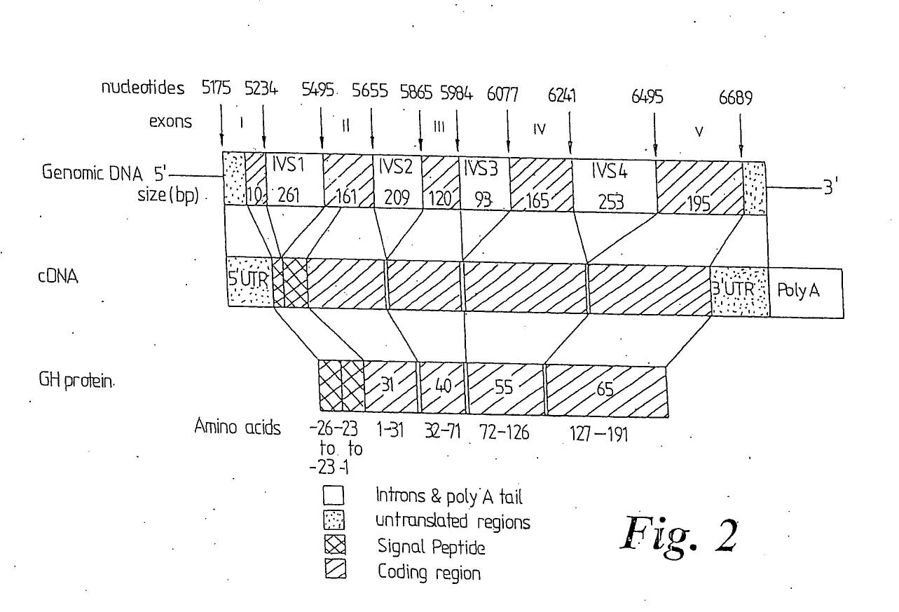

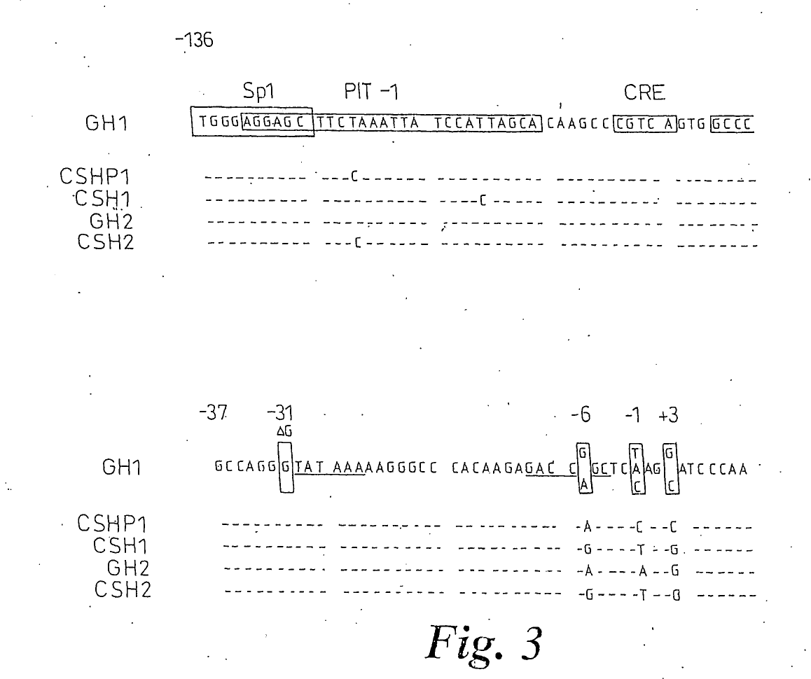

The gene encoding pituitary growth hormone (GH1) is located on chromosome 17q23 within a cluster of five related genes (FIG. 1). This 66.5 kb cluster has now been sequenced in its entirety [Chen et al. Genomics 4 479-497 (1989) and see FIG. 4]. The other loci present in the growth hormone gene cluster are two chorionic somatomammotropin genes (CSH1 and CSH2), a chorionic somatomammotropin pseudogene (CSHP1) and a growth hormone gene (GH2). These genes are separated by intergenic regions of 6 to 13 kb in length, lie in the same transcriptional orientation, are placentally expressed and are under the control of a downstream tissue-specific enhancer. The GH2 locus encodes a protein that differs from the GH1-derived growth hormone at 13 amino acid residues. All five genes share a very similar structure with five exons interrupted at identical positions by short introns, 260 bp, 209 bp, 92 bp and 253 bp in length in the case of GH1 (FIG. 2).

Exon 1 of the GH1 gene contains 60 bp of 5′ untranslated sequence (although an alternative transcriptional initiation site is present at −54), codons −26 to −24 and the first nucleotide of codon −23 corresponding to the start of the 26 amino acid leader sequence. Exon 2 encodes the rest of the leader peptide and the first 31 amino acids of mature GH. Exons 3-5 encode amino acids 32-71, 72-126 and 127-191, respectively. Exon 5 also encodes 112 bp 3′ untranslated sequence culminating in the polyadenylation site. An Alu repetitive sequence element is present 100 bp 3′ to the GH1 polyadenylation site. Although the five related genes are highly homologous throughout their 5′ flanking and coding regions, they diverge in their 3′ flanking regions.

A number of investigations have been undertaken on the GH1 gene and as a result of same known polymorphisms in the human GH1 gene promoter/5′ of the five untranslated regions have been identified and are as detailed in our co-pending patent application WO 03/042245. Additionally, other investigations have documented gross deletions in the GH1 gene, micro deletions in the GH1 gene and single base pair substitutions. All these variants of the GH1 gene are documented in our co-pending patent application WO 03/042245 and the skilled reader is therefore referred to this patent specification for more background information concerning the nature of GH1 variants that exist.

Since most cases of familial GH deficiency hitherto described are inherited as an autosomal recessive trait, some examples of the inherited deficiency state are likely to have gone unrecognized owing to small family size. Similarly, cases of GH deficiency resulting from de novo mutations of the GH1 gene could be classified as sporadic, and a genetic explanation for the disorder would neither be entertained nor sought. Finally, depending upon the criteria used for defining the deficiency state, it may be that the full breadth of both the phenotypic and genotypic spectrum of GH deficiency may never have come to clinical attention. For these reasons, current estimates of the prevalence of GH deficiency could be inaccurate and may therefore seriously underestimate the true prevalence in the population.

We have therefore investigated further the GH1 gene. As a result of our investigations we have identified a novel and significant variant that has implications for GH diagnosis and treatment. We consider that the identification of our novel variant indicates that GH deficiency is being under-diagnosed due to our current dependence on radio-immunoassay-based GH “function tests”. Further, it demonstrates an urgent need for the development of a true functional diagnostic assay.

Accordingly, the present invention provides an isolated variant of the growth hormone nucleic acid molecule, GH1, comprising the following substitution: +1491 C→G wherein 1491 refers to the position of the nucleotide with respect to the transcription initiation site which is designated 1.

According to a further aspect of the invention there is provided an isolated variant of the growth hormone nucleic acid molecule, GH1, comprising a nucleic acid molecule that encodes a protein, i.e. a GH protein, including the substitution Ile179Met.

In particular, the present invention provides a nucleic acid sequence as defined above, wherein the sequence is a DNA or RNA sequence, such as cDNA or mRNA.

The present invention therefore also provides a transcript of variant GH1, such as a protein (hereinafter ‘GH variant’) comprising an amino acid sequence encoded by said variant of GH1.

According to a further aspect of the invention there is therefore provided an isolated polypeptide which is a variant of the growth hormone protein, GH, and which includes the substitution Ile179Met.

Unexpectedly, our studies have shown that we have identified a variant GH which, uniquely, differentially activates receptor-mediated cell signalling pathways. Not only is an agonist acting in this fashion unprecedented, it is also extremely important in terms of the detection and treatment of growth hormone deficiency. It means that tests to identify growth hormone deficiency should, amongst other things, focus on the ability of circulating (or endogenous) growth hormone to activate more than one receptor-mediated cell signalling pathway. Our investigations of this detail reveal the full efficacy of circulating growth hormone and, correspondingly, the nature or identification of any growth hormone deficiency.

It follows that an investigation of growth hormone deficiency that does not focus on the identification of the above variant, or the differential activation of a receptor-mediated cell signalling pathway, will fail to identify the potential lack of activity of circulating growth hormone and thus growth hormone deficiency.

Accordingly, the present invention provides a screening method for screening an individual suspected of having dysfunctional GH, which screening method comprises the steps of:

(a) obtaining a test sample comprising a nucleic acid molecule of the human GH1 gene from the individual;

(b) sequencing said molecule;

(c) examining said sequence for a +1491C→G substitution; and

(d) where said substitution exists concluding there is a GH dysfunction.

Preferably, the test sample comprises genomic DNA, which may be extracted by conventional methods.

In the screening method of the invention, the sequencing step may be carried out in conventional manner, for example by PCR sequencing the appropriate region of the GH1 gene.

Accordingly also the present invention provides a screening method for screening an individual suspected of having dysfunctional GH, which screening method comprises the steps of:

(a) obtaining a test sample comprising a growth hormone, GH, polypeptide from said individual;

(b) sequencing said polypeptide;

(c) examining said sequence for a Ile179Met substitution; and

(d) where said substitution exists concluding there is a GH dysfunction.

The above screening methods involve a single blood test that can be performed in a clinic and provides for the early diagnosis of functional GH deficiency. This early diagnosis means that GH treatment can be started early and so reduce any of the harmful effects of GH dysfunction.

Accordingly, the present invention further provides a kit suitable for use in carrying out the screening method of the invention, which kit comprises:

(a) an oligonucleotide having a nucleic acid sequence corresponding to region +1491 of a GH1 gene, which region comprises the substitution +1491C→G; and

(b) an oligonucleotide having a nucleic acid sequence corresponding to the wild-type sequence in the region specified in (a); and, optionally,

(c) one or more reagents suitable for carrying out PCR for amplifying desired regions of the patient's DNA.

Such reagents may include, for example, PCR primers corresponding to the exon of the GH1 gene containing nucleotide +1491, and/or primers defined herein; and/or other reagents for use in PCR, such as Taq DNA polymerase.

Preferably, the primers or oligonucleotides in the kit comprise in the range of 20 to 25 base-pairs, such as 20 base-pairs for the variant sequence and 20 for the wild-type. In any case, the oligonucleotides must be selected so as to be unique for the region selected and not repeated elsewhere in the genome.

Other nucleotide detection methods could be used, such as signal amplification methods being pioneered in nanotechnology (such as Q-Dots). Also, single molecule detection methods could be employed (such as STM). In which case, the kit according to this invention may comprise one or more reagents for use in such alternative methods.

Alternatively, the screening method and corresponding kit according to this invention may be based on one or more so-called ‘surrogate markers’ that are indicative of, or correlated to, the presence of a variant of GH1 or a GH variant, such as proteins/amino acid sequences eg antibodies specific for a GH variant or a variant of GH1. Such a “surrogate marker” may comprise:

(a) any biomolecule (including, but not limited to, nucleotides, proteins, sugars, and lipids);

(b) a chemical compound (including, but not limited to, drugs, metabolites thereof, and other chemical compounds); and/or

(c) a physical characteristic,

whose absence, presence, or quantity in an individual is measurable and correlated with the presence of a GH variant or a variant of GH1 according to the present invention.

Further, suitable, alternative screening methods according to this invention may further comprise obtaining a test sample comprising a GH variant (ie a protein/peptide sequence comprising the Ile179Met variation of hGH) that is identifiable by conventional protein sequence methods (including mass spectroscopy, micro-array analysis, pyrosequencing, etc), and/or antibody-based methods of detection (eg ELISA), and carrying out one or more such protein sequencing method(s).

In which alternative cases, the kit according to this invention may comprise one or more reagents for use in such alternative methods.

According to a yet further aspect of the invention there is provided an isolated growth hormone polypeptide or protein which contains the Ile179Met substitution and which further provides for differential activation of receptor-mediated cell signalling pathways.

In a preferred embodiment of the invention said isolated polypeptide or protein variant activates the STAT5 pathway but shows reduced activation of the MAPK pathway.

In yet a further preferred embodiment of the invention said reduction in activity of the MAPK pathway is less than 70%, with respect to the activity of the wild type GH protein, and, more preferably still, is less than 50% and, more typically, 45% or less.

According to yet a further aspect of the invention there is provided an isolated growth hormone protein which is characterised by having an amino acid substitution in the C-terminal portion of helix 4. More preferably the substitution occurs at or adjacent the binding site for GHR residue Trp169 or Trp104.

According to a further aspect of the invention there is provided an isolated growth hormone polypeptide or protein which is characterised by possessing a reduced ability to activate the MAPK pathway.

In a preferred embodiment of the invention said MAPK pathway is an ERK pathway.

According to a further aspect of the invention there is provided a screening method for screening an individual suspected of having dysfunctional GH which screening method comprises the steps of:

(a) obtaining a test sample from said individual, which sample includes the individual's endogenous growth hormone;

(b) examining said growth hormone to determine whether and to what extent it will activate the receptor-mediated MAPK cell signalling pathway; and

(c) where there is a reduction in MAPK cell signalling, with respect to wild-type GH, concluding there is a GH dysfunction.

According to a further aspect of the invention there is, provided the use of the GH1 (nucleic acid) variant or GH (polypeptide/protein) variant as afore described for diagnosis of growth hormone dysfunction or the development of suitable GH therapeutics.

According to a yet further aspect of the invention there is provided an antibody specific for the isolated growth hormone polypeptide or protein of the invention.

The present invention further provides a composition comprising a GH1 or GH variant of this invention in association with a pharmaceutically acceptable carrier therefor.

Furthermore, the invention provides:

(a) a vector comprising a nucleic acid molecule according to the present invention;

(b) a host cell comprising the vector (a), such as a bacterial host cell; and

(c) a process for preparing a GH variant according to this invention, which process comprises:

(i) culturing the host cell (b); and

(ii) recovering from the culture medium the GH variant thereby produced.

(d) a protein or amino acid sequence being in culture medium and encoded or expressed by a sequence, vector, or cell as defined above.

The present invention will now be illustrated with reference to the following Examples.

FIG. 1 shows the location of GH1 gene, on chromosome 17q23, with respect to the four paralogous genes i.e. CSHP1, CSH1, GH2 and CSH2;

FIG. 2 is a detailed illustration of the GH1 gene showing the introns, exons untranslated regions, signal peptide, coding region and poly A tail;

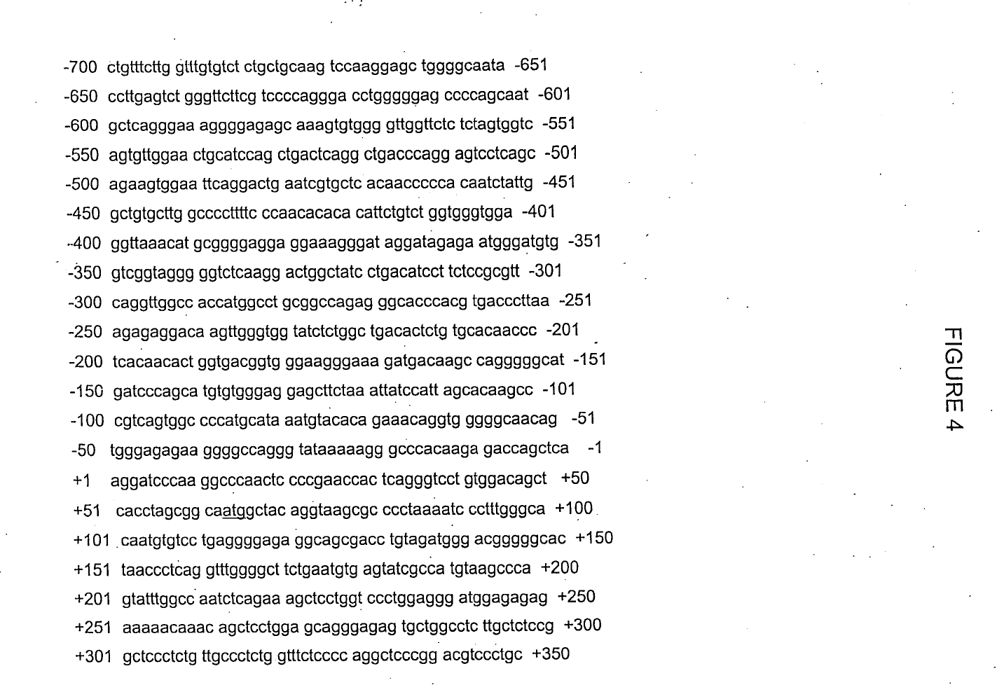

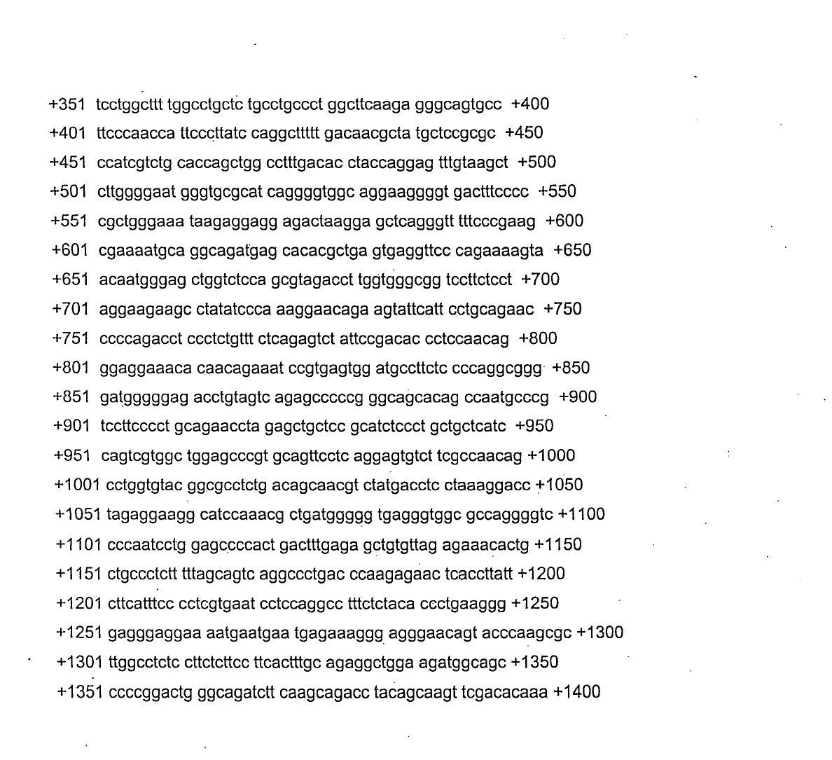

FIG. 3 shows the promoter of the GH1 and the very high level of sequence polymorphism associated therewith;

FIG. 4 shows the reference nucleic acid sequence structure of the GH1 gene;

FIG. 5 shows the nucleic acid coding sequence of the GH1 gene and the corresponding polypeptide sequence;

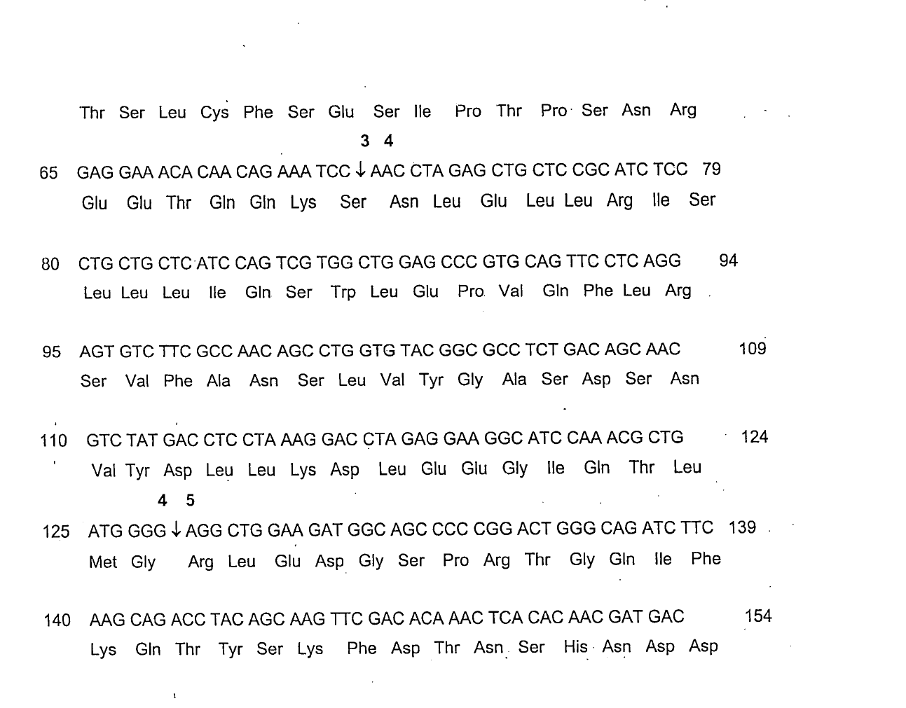

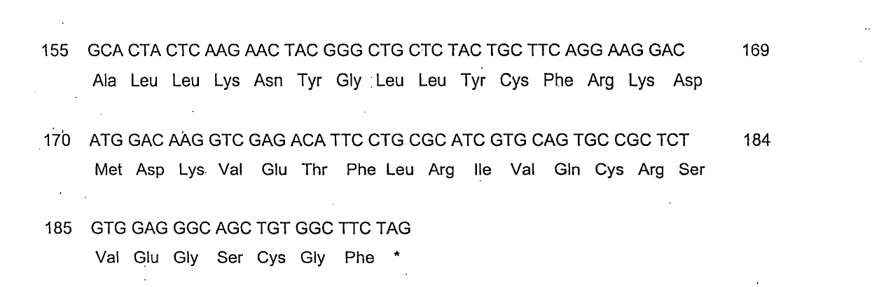

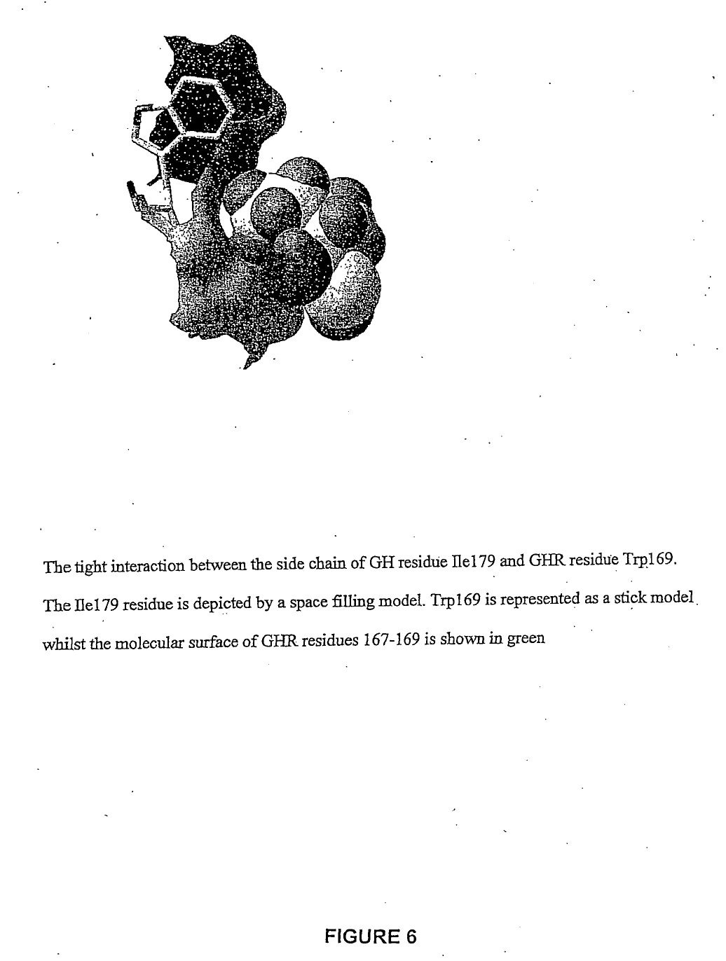

FIG. 6 is an illustration of the molecular modelling of growth hormone protein when interacting with its corresponding receptor. The illustration shows the tight interaction between the side chain of GH residue Ile179 and GHR residue Trp169. The Ile179 residue is depicted by a space filling model. Trp169 is represented as a stick model whilst the molecular surface of GHR residues 167-169 is shown in green;

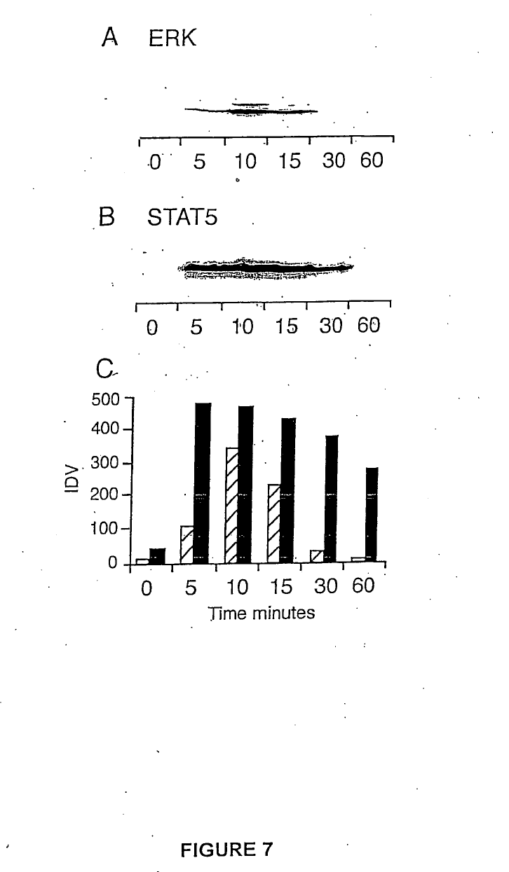

FIG. 7 shows the time course of activation of ERK and STAT 5. Data shown are western blots probed with phospho-specific. ERK and STAT 5 antibodies showing time-dependent activation of ERK (A) and STAT 5 (B). The ERK blots show the upper fainter band corresponding to ERK 1 and the lower dark band corresponding to ERK 2. The blots were analysed by imaging densitometry and the data (integrated density values, IDV) were normalised for total ERK or STAT 5 and plotted against GH treatment time (C) showing that ERK activation (hatched columns) peaked at 10 minutes and STAT 5 (solid columns) peaked at 5-10 minutes following GH treatment;

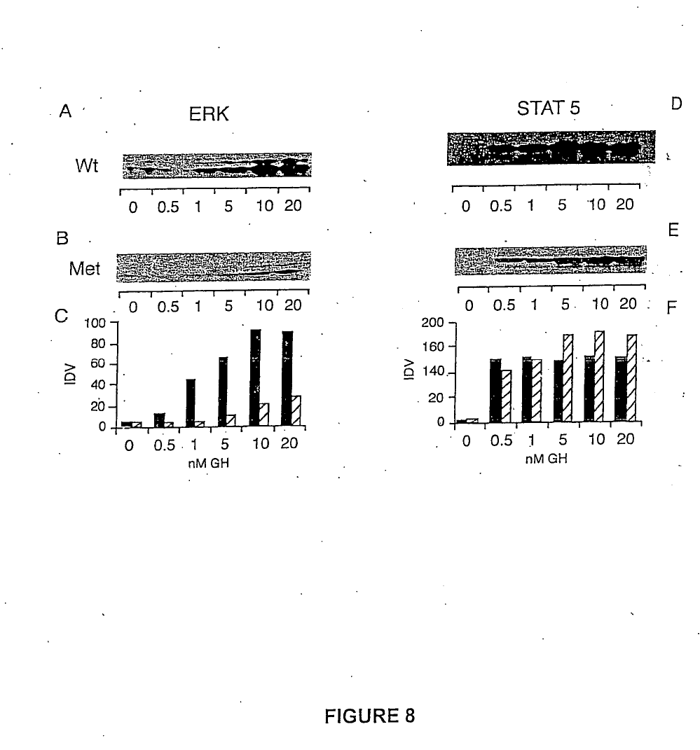

FIG. 8 shows western blot analysis of dose-dependent (0.5-20 nM) activation of ERK (A-C) and STAT 5 (D-F) by wild-type (Wt, A, d) and Ile179Met GH (Met, B, E). Blots were analysed by imaging desitometry and the data (integrated density value, IDV) normalised for total ERK and STAT 5 showing reduced activation of ERK (C) by the GH variant (hatched columns) compared to wild-type GH (solid columns) at all doses studied, this contrasted with similar activation of STAT 5 (F) by both variant (hatched columns) and wild-type GH (solid columns); and

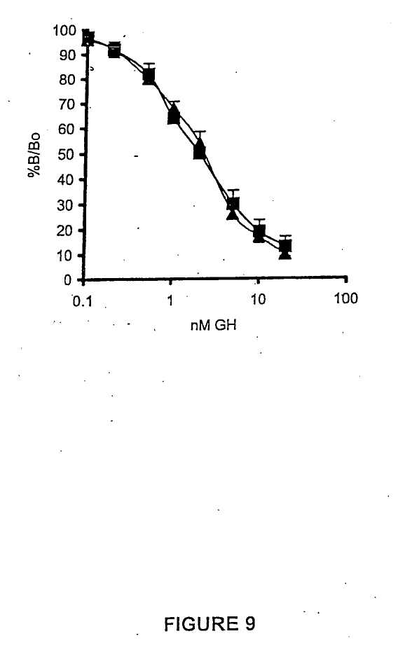

FIG. 9 shows GHR binding characteristics of wild-type (triangles) and Ile179Met variant GH. Data are expressed as displacement of specific ¹²⁵I-labelled GH binding (% B/Bo) over a dose range of 0.1-20 nM of unlabelled wild-type and variant GH. Each point is the mean of 4 separate experiments ±SEM.

### EXAMPLE 1

### Patient Selection—Andalucia/Barcelona Study

A different patient cohort was established in Andalucia, Spain. Seventy-four pre-pubertal children were selected on the basis of their classification as FSS, ie exhibiting familial short stature, as defined by Ranke in Hormone Research 45 [(Suppl. 2) 64-66 (1996)]. Such patients have at least one genetic family member exhibiting short stature. The height deviation score (SDS) of all the children in the study was −2 SDS below the mean for the general population. All subjects exhibited normal GH secretion after a pharmacological stimulation test (peak GH values ≧10 ng/mL). Pharmacological tests used were clonidine (34 cases), propanolol (25 cases) and insulin (15 cases). Ethical approval for genetic studies was obtained from each participating centre and the Multi-Regional Ethics Committee. Written informed consent was obtained from each participating individual.

Standard deviation scores were calculated for height, body mass index, paternal and maternal heights, mid-parental height, IGF-1 and IGFBP-3 levels, peak GH secretion in ng/mL, and GHBP (as a percentage). These data are presented in Table 2 for the two individuals (B4 and B49) in whom a novel GH1 gene lesion was found and as group means for the cohort of individuals studied.

TABLE 2
Auxological parameters and laboratory investigations for the individuals
with novel GH1 mutations as compared to group means.

| | | | Group |
|---|---|---|---|
| Patient | B49 | B4 | mean (SD) |
| measurement | (Ile179Met) | (−360 A G) | n = 74 |
| Chronological | 6.9 | 6.0 | 8.6 (2.2) |
| age (years)* | | | |
| Bone age (years) | 6.4 | 6.6 | 8.0 (2.5) |
| Height (cm) | 113 | 112.5 | — |
| Height (SDS) | −2.7 | −2.1 | −2.4 (0.6) |
| Height velocity | −1.6 | −0.6 | −1.1 (1.0) |
| (SDS) | | | |
| Weight (kg) | 19.0 | 17.8 | — |
| Body mass | 0.4 | 0.1 | −0.5 (1.0) |
| index (SDS) | | | |
| Maternal | −3.9 | −2.3 | — |
| height (SDS) | | | |
| Paternal | −1.4 | −1.7 | — |
| height (SDS) | | | |
| Mid-paternal | −2.7 | −2.0 | — |
| height (SDS) | | | |
| IGF-1 (SDS) | −1.2 | −1.7 | −0.9 (1.2) |
| IGFBP-3 (SDS) | 1.5 | 0.4 | 0.1 (1.3) |
| GH peak (ng/mL) | 10.4 | 16.8 | 17.6 (9.4) |
| | (propanolol + | (clonidine) | |
| | exercise) | | |
| GHBP (%) | 27.5 | 29.8 | 27.8 (5.7) |

*All auxology data taken at this age.

### EXAMPLE 2

### Polymerase Chain Reaction (PCR) Amplification of a GH1-Specific Fragment

PCR amplification of a 3.2 kb GH1-specific fragment has been performed on the patients selected as per Example 1 and controls. Genomic DNA was extracted from patient lymphocytes by standard procedures.

Oligonucleotide primers GH1F (5′ GGGAGCCCCAGCAATGC 3′; −615 to −599) and GH1R (5′ TGTAGGAAGTCTGGGGTGC 3′; +2598 to +2616) were designed to correspond to GH1-specific sequences in order to PCR amplify a 3.2 kb single genomic DNA fragment containing the human GH1 gene using the Expand™ high fidelity system (Roche).

Two separate thin-walled 0.65 ml PCR tubes were used for each reaction. The first tube contained 500 nanograms (ng) each primer (GH1F and GH1R), 200 μM dATP, dTTP, dCTP and dGTP and 200 ng of patient genomic DNA made up to a final volume of 25 μl with sterile water. The second tube contained 5 μl 10× reaction buffer made up to a final volume of 24.25 μl with sterile water. Both tubes were placed on ice for 5 minutes. After this time, 0.75 μl of Expand™ polymerase mix was added to the second tube, the contents mixed and transferred to the first tube. The tube was centrifuged for 30 seconds and the reaction mixture overlaid with 30 μl light mineral oil (Sigma). The reaction mixture was then placed in a 480 or 9700 PCR programmable thermal cycler (Perkin Elmer) set at 95° C.

The reaction mix was then amplified under the following conditions: 95° C. for 2 minutes followed by 30 cycles of 95° C. for 30 seconds, 58° C. for 30 seconds and 68° C. for 2 minutes. For the last 20 cycles, the elongation step at 68° C. was increased by 5 seconds per cycle. This was followed by a further incubation at 68° C. for 7 minutes and the reaction was then cooled to 4° C. prior to further analysis. For each set of reactions, a blank (negative control) was also set up. The blank reaction contained all reagents apart from genomic DNA and was used to ensure that none of the reagents were contaminated.

A one-tenth volume (5 μl) was analysed on a 1.5% agarose gel to assess whether PCR amplification had been successful before nested PCR was performed. Those samples that had PCR-amplified successfully were then diluted 1 in 100 prior to use for nested PCR.

Further, in our study additional primers used for sequencing in the reverse direction were GHBFR (5′ TGGGTGCCCTCTGGCC 3′; −262 to −278), GHSEQ1R (5′ AGATTGGCCAAATACTGG 3′; +215 to +198), GHSEQ2R (5′ GGAATAGACTCTGAGAAAC 3′; +785 to +767), GHSEQ3R (5′ TCCCTTTCTCATTCATTC 3′; +1281 to +1264), GHSEQ4R (5′ CCCGAATAGACCCCGC 3′; +1745 to +1730) [Numbering relative to the transcriptional initiation site at +1; GenBank Accession No; J03071]. Samples containing sequence variants were cloned into pGEM-T (Promega, Madison Wis.) followed by sequencing of a minimum of four clones per individual.

### EXAMPLE 3

### Nested-PCR

Nested PCR was performed on the fragments produced in Example 2 to generate, in each case, seven overlapping sub-fragments that together span the entire GH1 gene. In addition, the Locus Control Region has been PCR-amplified (see Example 5) in all but three patients.

The seven overlapping sub-fragments of the initial 3.2 kb PCR product were PCR-amplified using Taq Gold DNA polymerase (Perkin-Elmer). Oligonucleotides used for these reactions are listed in Table 6 together with their sequence locations as determined from the GH1 gene reference sequence.

A 1 μl aliquot of the diluted long (3.2 kb) PCR product was put into a thin-walled 0.2 ml PCR tube or into one well of a 96-well microtitre plate. To this was added 5 μl 10× reaction buffer, 500 ng appropriate primer pair (e.g. GH1DF and GH1DR), dATP, dTTP, dCTP and dGTP to a final concentration of 200 μM, sterile water to a volume of 49.8 μl, followed by 0.2 μl Taq Gold polymerase.

The tube or microtitre plate was then placed in a Primus 96 thermal cycler (MWG Biotech) and cycled as follows: 12 min 95° C. followed by 32 cycles of 95° C. for 30 seconds, 58° C. for 30 seconds and 72° C. for 2 minutes. This was followed by further incubation at 72° C. for 10 minutes and the reaction was then cooled to 4° C. prior to further analysis.

A one-tenth volume (5 μl) of the reaction mix was analysed on a 0.8% agarose gel to determine that the reaction had worked before denaturing high-pressure liquid chromatography (DHPLC) was performed on a WAVE™ DNA fragment analysis system (Transgenomic Inc. Crewe, Cheshire, UK). To enhance heteroduplex formation, the PCR product was denatured at 95° C. for 5 minutes, followed by gradual re-annealing to 50° C. over 45 minutes. Products were loaded on a DNAsep column (Transgenomic Inc.) and eluted with a linear acetonitrile (BDH Merck) gradient of 2%/min in a 0.1M triethylamine acetate buffer (TEAA pH 7.0), at a constant flow rate of 0.9 ml/minute. The start and end points of the gradient were adjusted according to the size of the PCR product. Analysis took 6.5-8.5 minutes per amplified sample, including the time required for column regeneration and equilibration. Samples were analysed at the Melt temperatures (TM) determined using the DHPLCMelt software (http://insertion.stanford.edu/melt.html) and listed in Table 3. Eluted DNA fragments were detected by an UV-C detector (Transgenomic Inc.).

TABLE 3

Oligonucleotide primers used for DHPLC analysis
and DNA sequencing

| | | | | DHPLC |
|---|---|---|---|---|
| | | | | melt |
| Frag- | | | | temper- |
| ment | Primer | Sequence (5′ to 3′) | Position | ature |
| 1 | CH1DF | CTCCGCGTTCAGGTTGGC | −309 to −292 | 60° C. |
| | GH1DR | CTTGGGATCCTTGAGCTGG | −8 to +11 | |
| 2 | GH2DF | GGGCAACAGTGGGAGAGAAG | −59 to −40 | 63° C. |
| | GH2DR | CCTCCAGGGACCAGGAGC | +222 to +239 | |
| 3 | GH3DF | CATGTAAGCCCAGTATTTGGCC | +189 to +210 | 62° C. |
| | GH3DR | CTGAGCTCCTTAGTCTCCTCCTCT | +563 to +586 | |
| 4 | GH4DF | GACTTTCCCCCGCTGGGAAA | +541 to +560 | 62° C. |
| | GH4DR | GGAGAAGGCATCCACTCACGG | +821 to +841 | |
| 5 | GH5DF | TCAGAGTCTATTCCGACACCC | +772 to +792 | 62° C. |
| | GH5DR | GTGTTTCTCTAACACAGCTCTC | +1127 to +1148 | |
| 6 | GH6DF | TCCCCAATCCTGGAGCCCCACTGA | +1099 to +1122 | 62° C. |
| | GH6DR | CGTAGTTCTTGAGTAGTGCGTCATCG | +1410 to +1435 | |
| 7 | GH7DF | TTCAAGCAGACCTACAGCAAGTTCG | +1369 to +1393 | 57° C. |
| | | | | and |
| | | | | 62° C. |
| | GH7DR | CTTGGTTCCCGAATAGACCCCG | +1731 to +1752 | |

With respect to the samples obtained from patients selected according to Example 1A above, the following procedures (Examples 4 & 5) were carried out:

### EXAMPLE 4

### DNA-Sequencing of GH1-Specific Long PCR Fragments

Clones containing the GH1-specific long PCR fragment were sequenced with the BigDye (RTM) sequencing kit (Perkin Elmer) in either 0.2 ml tubes or 96-well microtitre plates in a Primus 96 (MWG) or 9700 (Perkin Elmer) PCR thermal cycler. Oligonucleotide primers used for sequencing were:

GH1S1
(5′ GTGGTCAGTGTTGGAACTGC 3′: −556 to −537);
GH3DF
(5′ CATGTAAGCCAAGTATTTGGCC 3′: +189 to +210);
GH4DF
(5′ GACTTTCCCCCGCTGTAAATAAG 3′: +541 to +560):
and
GH6DF
(5′ TCCCCAATCCTGGAGCCCCACTGA 3′: +1099 to +1122).

1 μg of cloned DNA was sequenced with 3.2 pmol of the appropriate primer and 4 μl BigDye sequencing mix in a final volume of 20 μl. The tube or microtitre plate was then placed in the thermal cycler and cycled as follows: 2 minutes 96° C. followed by 30 cycles of 96° C. for 30 seconds, 50° C. for 15 seconds and 60° C. for 4 minutes. The reaction was then cooled to 4° C. prior to purification.

Purification was performed by adding 80 μl 75% isopropanol to the completed sequencing reaction. This was then mixed and left at room temperature for 30 minutes. The reaction was then centrifuged at 14,000 rpm for 20 minutes at room temperature. The supernatant was then removed and 250 μl 75% isopropanol was added to the precipitate. The sample was mixed and centrifuged for 5 minutes at 14,000 rpm at room temperature. The supernatant was removed and the pellet dried at 75° C. for 2 minutes.

Samples were then analysed on an ABI Prism 377 or 3100 (Applied Biosystems) DNA sequencer.

### EXAMPLE 5

### GH1 Gene Mutations and Polymorphisms

Three mutations of potential pathological importance were found in the sequence analysis of the 74 familial short stature patients from Barcelona: −360 A→G (Patient B4), GTC→ATC at +1029 (Val 110→Ile) (Patient B53; this variation is also described in co-pending patent specification no. PCT/GB01/2126) and ATC→ATG at +1491 (Ile179→Met) (Patient 49). See Table 4.

Since four Ile110 alleles were noted in the control sample (a frequency of 0.025), this variant occurs at polymorphic frequencies in the general population. Molecular modelling suggested that this substitution might exert a deleterious effect on the structure of GH; the evolutionarily-conserved Val110 residue forms part of the hydrophobic core at the N-terminal end of helix 3, and its replacement by Ile with its longer sidechain would be expected to cause steric hindrance. Consistent with this prediction, the Ile110 variant is associated with a dramatically reduced ability (40% of normal) to activate the JAK/STAT signal transduction pathway. The Val110→Ile substitution appears therefore to represent a functional polymorphism that is associated with a reduction in GH activity and which is potentially able to influence stature. This variation is associated with promoter haplotype 2, which has fairly normal activity.

With respect to the Ile179Met variation: Ile179 is positioned at the surface of the hGH protein centrally in helix 4. In the hGHbp/hGH 2:1 complex, Ile179 interacts directly with the ‘hot-spot’ residues of site 1, TRP104 and TRP169. It is therefore likely that a substitution of Ile179 with a methionine residue would interfere with a precise steric constraint in site 1, thereby affecting signal transduction and resulting in a significant change in the functioning of the hGH.

(c) Studies of GH1 Coding Sequence Variation in Controls

A total of 80 healthy British controls of Caucasian origin were also screened for variants, using the method of Examples 2 and 3, within the coding region of the GH1 gene. Five examples of silent substitutions found in single individuals were noted [GAC→GAT at Asp26, TCG→TCC at Ser85, TCG→TCA at Ser85, ACG→ACA at Thr123 and AAC→AAT at Asn109]. The Thr123 polymorphic variant has been reported previously (Counts et al Endocr Genet 2 55-60 (2001)).

In addition, three missense substitutions were noted [ACC→ATC, Thr271→Ile; AAC→GAC, Asn47→Asp; GTC→ATC, Val110→Ile, 1, 1 and 4 alleles respectively/160 alleles]; only the Val110→Ile substitution had been found in the patient study disclosed in our co-pending patent specification no. PCT/GB01/2126 (patient 66). Molecular modelling suggested that this substitution might exert a deleterious effect on the structure of GH; Val110 forms part of the hydrophobic core at the N-terminal end of helix 3 and its replacement by Ile with its longer sidechain would cause steric hindrance. It may thus be that while the Val110→Ile substitution in both control and patient populations, it is nevertheless capable of influencing stature. Other comments apply as in Example 5(b) above. This notwithstanding, the relative paucity of missense mutations in the control population argues in favour of the pathological significance of the lesions found in the patient cohort.

### EXAMPLE 6

### Biological Activity of the Ile179Met Variant

HK293 cells, transfected with the full-length human GH receptor (GHR) and selected on the basis of elevated GHR expression (HK293Hi cells), were used to assay the biological activity of the GH variant [Ross et al Mol Endocrinol 11 265-73 (1997); von Laue et al J Endocrinol 165 301-311. (2000)]. Cells were plated into 24-well plates (100,000 cells per well) for 24 hrs in DMEM:F-12 (1:1) containing 10% FCS. Cells were co-transfected overnight using a lipid-based transfection reagent (FuGENE 6, Roche Molecular Biochemicals) with a STAT5-responsive luciferase reporter gene construct [Ross et al, ibid, von Laue et al, ibid] and a constitutively expressed β-galactosidase expression vector (pCH110; Amersham Pharmacia Biotech) to correct for transfection efficiency. Cells were then incubated with variant and wild-type GH diluted to a known standard range of concentrations (0.1-10 nM) in serum-free DMEM:F-12 (1:1) containing 2.5×10⁻⁷M dexamethasone for 6 hours to allow GHR dimerization, STAT5 activation and luciferase expression. After incubation, cells were lysed and the luciferase measured in a microplate luminometer (Applied Biosystems) using the Promega luciferase assay system. Luciferase expression thus provided a measure of the degree of GHR activation and hence the biological activity of the GH variant. Experiments were carried out in quadruplicate and repeated at least 3 times. Statistical analysis of luciferase assay data was carried out by analysis of variance (ANOVA) with subsequent comparisons using the Student-Newman-Keuls multiple comparison test.

### EXAMPLE 7

### GH Secretion Studies in Mammalian Cells

The rat pituitary (GC) cell line was transfected with a pGEM-T plasmid containing a 3.2 kb fragment spanning the entire wild-type GH1 gene (under the control of promoter haplotype 1) and equivalent constructs for the missense variant under the control of their associated haplotypes. Cells were plated into 24-well plates (200,000 cells per well) and cultured overnight in DMEM containing 15% horse serum and 2.5% FCS (complete medium). Cells were co-transfected with 500 ng GH1 plasmid and β-galactosidase expression vector (pCH110; Amersham Pharmacia Biotech) using the lipid-based transfection reagent Tfx-20 (Promega). Transfection was carried out in 200 μl serum-free medium containing 1 μl Tfx-20/well for 1 hr, after which 0.5 ml complete medium was added to each well. Cells were cultured for 48 hrs, medium harvested and cells lysed for β-galactosidase assay to correct for differences in transfection efficiency. GH in the medium was quantified for the variant using a human GH IRMA (Nichols Institute Diagnostics) that showed no cross-reactivity with rat GH. Experiments were performed and data analysed as described for the biological activity assay.

### EXAMPLE 8

### Functional Characterization of Missense Variant

Missense mutation in the mature protein was modelled by simple replacement of the appropriate amino acid residue in the X-ray crystallographic structure of human GH.

Molecular Modelling

The Ile179Met variant was structurally analysed by inspection of the appropriate amino acid residue in the X-ray crystallographic structure of human GH (PDB: 3HHR) [19]. The wild-type and mutant GH structures were compared with respect to electrostatic interactions, hydrogen bonding, hydrophobic interactions and surface exposure. Molecular graphics were performed using the ICM molecular modelling software suite (Molsoft LLC, San Diego, Calif.). (FIG. 6)

### EXAMPLE 9

### Proteolytic Digestion of the GH Variant

Trypsin, chymotrypsin, or proteinase K (all Sigma, Poole, UK) were added to a final concentration of 0.1 μg/ml to 100 μl culture medium harvested from insect cells expressing either wild-type GH or the Ile179Met variant (60 nM) and then incubated at 37° C. for 1 hr. Previous dose-dependent studies on wild-type GH indicated that 0.1 μg/ml was the concentration at which degradation was initiated by all three enzymes. After the 1 hr treatment period, 10 μl trypsin-chymotrypsin inhibitor (500 μg/ml) was added to stop the trypsin and chymotrypsin digests and 1 μl PMSF (0.1M) was added to stop the proteinase K digest. Each reaction was then incubated for a further 15 mins at 37° C. The samples were analysed by SDS-PAGE on a 12% gel using a mini gel apparatus (Bio-Rad Laboratories). An equivalent amount of undigested wild-type GH and Ile179Met variant that had been incubated for 1 hr at 37° C. was also run on the gel. The gel was electroblotted onto PVDF membrane as previously described [Lewis et al J Neuroendrocinol14 361-367 (2002)], probed with a mouse monoclonal anti-human GH antibody (Lab Vision, Fremont, Calif., USA), diluted 1:500, detected using an anti-mouse IgG-HRP conjugate (1:5000, Amersham Biosciences) and visualised by enhanced chemiluminescence (ECL Plus, Amersham Biosciences). Films were analysed using the Alpha Imager 1200 digital imaging system (Alpa Innotech Corp, San Leandro, Calif., USA) and the results expressed as the amount of GH remaining following enzyme digestion as a percentage of undigested GH. The experiments were repeated 3 times and assessed statistically by a two-tailed t-test.

### EXAMPLE 10

### Activation of the MAP Kinase Pathway

The ability of the Ile179Met variant to activate the MAP kinase signal transduction pathway to the same degree as wild-type GH was investigated by stimulating 3T3-F442A preadipocytes with wild-type GH and the Ile179Met variant. Cells (250,000) were plated into 10 cm culture dishes and cultured in DMEM containing 10% calf serum for three days prior to the experiment. The plates were washed with PBS and the cells incubated in serum-free DMEM for two successive 2-hour wash-out periods. GH was spiked directly into the serum-free DMEM at the end of the second wash-out period and the cells incubated for the appropriate time. After this period, the medium was removed and the cells were washed with ice cold PBS containing 1 mM sodium orthovanadate, lysed in 0.5 ml Laemmli buffer containing 1 mM orthovanadate and 1 mM PMSF and analysed by SDS-PAGE on a 10% gel as described above. The gel was blotted onto PVDF membrane and probed using antibodies that detect the activated (phosphorylated) forms of p42/p44 MAP kinase phosphorylated on residues Thr202/Tyr204 (Cell Signaling Technology) and STAT 5 phosphorylated on residues Tyr694/Tyr699 (Upstate Biotechnology). Blots were processed, visualised using ECL Plus (Amersham) and the images analysed as described above. To ensure equal protein loading between lanes, blots were stripped and reprobed with antibodies that recognise total MAPK or STAT 5 (Santa Cruz Biotechnology, Santa Cruz, Calif.) as appropriate. Both phospho-specific and total STAT 5 antibodies cross-react equally with STAT 5a and 5b. Second antibodies were either anti-mouse or anti-rabbit IgG-HRP conjugates depending on the primary antibody used (1:5000, Amersham Biosciences). Films were analysed by imaging densitometry as described above. Results for phospho-MAP and phospho-STAT 5 were normalised with respect to total MAP or STAT 5 in the same sample.

Initial studies were also performed to determine the time course of MAPK and STAT 5 activation by wild-type GH in our experimental model. STAT 5 activation by GH was rapid, peaking at 5-10 minutes with a gradual decline thereafter, whereas MAPK activation peaked at 10 minutes with a much more rapid decline, returning to basal levels of activation by 60 minutes (FIG. 7). A 10-minute GH treatment time was therefore selected for use in subsequent studies since this was the time of maximal MAPK activation and was on the plateau period of maximal STAT 5 activation. Cells were treated with a range of concentrations of wild-type and variant GH (0.5-2.0 nM) for 10 minutes and activation of MAPK and STAT 5 analysed (FIG. 8).

### EXAMPLE 11

### Functional Characterization of the Ile179Met Variant

The evolutionary conservation of the hydrophobic residue Ile179 was examined by ClustalW multiple sequence alignment of orthologous GH proteins from 19 vertebrates [Krawczak et al Gene 237 143-151 (1999)].

### EXAMPLE 12

### Receptor Binding Studies

Receptor binding studies were performed using HK293hi cells transfected with the full-length human GHR, and selected on the basis of elevated GHR expression (HK293hi cells) [Ross et al. Mol Endocrinol 11 265-273 (1997); von Laue et al., J. Endocrinol 165 301-311 (2000)]. 2 μg GH (human pituitary iodination grade, Calbiochem, San Diego, Calif., USA) were labelled with 37 MBq iodine-125 (Amersham Biosciences, Little Chalfont, Bucks, UK) to a specific activity of 87 MBq/nmole using chloramines T (0.7 mM) for 45 seconds and purified using a Sephadex G-10 column. Cells were plated into 12 well plates (300,000 per well) and cultured overnight in DMEM/F-12 (1:1) containing 10% fetal calf serum. Cultures were washed once in serum-free DMEM/F-12, pre-incubated in serum-free medium for 3 hours at 37° C., then washed twice with phosphate-buffered saline containing 1% bovine serum albumin (PBS-BSA) and incubated with labelled GH (200,000 cpm/well) in 1 ml PBS-BSA with varying amounts of either wild-type of Ile179Met GH for a further 3 hours at room temperature. At the end of the incubation period, cells were washed twice with PBS-BSA and solubilised in 1M NaOH for quantification of bound ¹²⁵I-GH. Experiments (n=4) were performed in duplicate wells and K_{d} values were calculated by Scatchard analysis of the data.

Results

Functional Characterization of the Ile179Met Variant

The evolutionary conservation of the hydrophobic residue Ile179 was examined by ClustalW multiple sequence alignment of orthologous GH proteins from 19 vertebrates [Krawczak et al Gene 237 143-151 (1999)]. This residue is a hydrophobic valine in all vertebrates except turtle, indicating that the substitution by Ile in the human lineage is conservative. Comparison with the paralogous genes of the human GH cluster revealed that the residue analogous to Ile179 is Met in CSH1, CSH2 and the CSH pseudogene (CSHP1). This is consistent with the conservative Ile179Met substitution having been templated by gene conversion.

The Ile179Met substitution was then modelled by replacement of the residue in the X-ray crystallographic structure of human GH. Ile179 lies in the C-terminal portion of helix 4, which is involved in site 1 binding and where it is partially exposed, allowing hydrophobic interactions with the side-chain of the “hotspot” GHR residue Trp169. Further interactions with the GHR occur between the side-chain and backbone atoms of Ile179 and the backbone atoms of GHR residues Lys167 and Gly168. Replacement of the Ile179 side-chain with the side-chain of methionine introduced unfavourable van der Waals (e.g. steric) interactions with the side chain of the Trp169 residue and indicates that these hydrophobic interactions may be conserved upon substitution. Receptor binding studies were performed to determine the affinity of wild-type GH and the Ile179Met variant for the GHR. The introduction of a methionine residue failed to alter the receptor binding (FIG. 9); the K_{d} values of the wild-type and Ile179Met GH molecules were found to be 1.99 nM and 2.04 nM respectively. This suggests that if any differences exist between the binding characteristics of wild-type and variant GH they are subtle and do not alter the K_{d} as measured in a static system. Alanine-scanning mutagenesis has previously identified Ile179Met as contributing to a patch of residues that determine receptor affinity; a non-conservative alanine substitution resulted in a marked decrease in GHR binding (K_{d} increased from 0.34 to 0.92 nM) [Cunningham and Wells, Science 244 1081-1084 (1989)]. In view of the fact that Ile179 contributes to receptor binding affinity, it may be that the Ile179Met variant exerts a subtle effect on GHR receptor binding.

We therefore investigated whether the perturbed interaction between the Ile179Met GH variant and GHR could lead to reduced activation of the STAT 5 and MAPK pathways. STAT 5 activation was studied indirectly using a luciferase reporter gene assay, and directly by determining the level of activated phospho-STAT 5 by Western blotting. MAPK activation was studied directly by determining the level of activated phospho-MAPK by Western blotting.

The Ile179Met variant was expressed in insect cells and a luciferase reporter gene assay system (11, 12) used to assay its signal transduction activity. For GH to be biologically active, it must bind to two GHR molecules thereby triggering receptor dimerization. GHR dimerization activates the intracellular tyrosine kinase JAK2 which in turn activates the transcription factor STAT 5 by phosphorylation. Phosphorylated STAT 5 dimerizes, translocates to the nucleus and binds to STAT 5-responsive promoters thereby switching on the expression of GH-responsive genes. The assay of GH biological activity used here requires all stages of this pathway to be functional. The Ile179Met variant was found to display normal (99±4% wild-type) ability to activate the JAK/STAT signal transduction pathway (FIG. 10) when compared to wild-type GH at a concentration of 1 nM, the approximate ED₅₀ value for GH in this assay system. Whilst this variant could simply have failed to manifest its detrimental effects in a static in vitro system, the possibility was also considered that it might have exerted its deleterious effects on a signal transduction pathway other than JAK/STAT. Alternatively, the Ile179Met substitution could compromise GH folding, secretion or stability in vivo, or have adverse effects on the GH axis that are as yet undefined.

Western blotting experiments using antibodies specific for the phosphorylated (activated) forms of MAPK and STAT 5 were undertaken to assess the ability of the Ile179Met variant to activate the MAP kinase and STAT 5 pathways. The Ile179Met variant exhibited a reduced ability to activate MAPK throughout the concentration range studied whereas there was no difference in STAT 5 activation (New FIG. 8). Analysis of the data from multiple experiments using a single maximal dose of GH (20 nM) indicated that the Ile179Met had a significantly reduced ability to activate MAPK to only 45% of the level produced by wild-type (4.35±0.66 times basal level of activation for the Ile179Met variant as compared to 9.76±2.52 time basal level of activation produced by wild-type GH; mean±SEM, n=5 separate experiments, p<0.05, Student's t-test, FIG. 8). This contrasted with the variant's ability to activate STAT 5 to the same level as wild-type GH at a concentration of 20 nM (36.50 times basal level for the wild-type versus 38.62 times for the Ile179Met variant). The Western blotting data confirmed the result from the STAT 5-responsive luciferase reporter gene assay showing similar levels of activity for both wild-type GH and the Ile179Met variant. By contrast, activation of the MAPK occurred at only half the level elicited by wild-type GH.

To explore these possibilities further, the secretion of the Ile179Met variant was studied in rat pituitary GC cells. The wild-type GH1 gene, under the control of GH1 promoter haplotype 1, was transfected into GC cells and shown to be responsible for the secretion of human GH (as measured by ELISA using a human GH-specific antibody) at a concentration of 64 μM over a 48 hr period. The level of secretion of the Ile179Met variant (also under the control of GH1 promoter haplotype 1 with which it is associated in cis in patient B49) was then assayed as previously described, and the GH secretion level measured was expressed as a percentage of wild-type. Since secretion was found to be 97±4% of the wild-type value, it may be inferred that this mutation is likely to have little or no effect on GH secretion.

Finally, the Ile179Met variant was also challenged with trypsin, chymotrypsin and proteinase K to determine if it was more susceptible to proteolytic cleavage than wild-type GH. However, the 179Met variant proved similarly resistant to proteolytic cleavage as wild-type GH indicating that there were no significant differences in protein folding between the two forms of GH. This should be considered within the context that some 67% of our previously identified GH variants [Millar et al, Hum Mutat 21 424-440 (2003)] manifested increased susceptibility to proteolysis as compared to wild-type GH. Consistent with the absence of misfolding, the level of secretion of the Ile179Met variant from rat pituitary cells was indistinguishable from that of wild-type GH.

To our knowledge, the differential activation of cell signalling pathways by a receptor agonist is quite unprecedented. In contrast to the Ile179Met variant, all GH variants previously identified in studies from our laboratory were either associated with reduced secretion or a reduced ability to activate the JAK/STAT pathway or both [Millar et al., Hum Mutat 21 424-440 (2003)]. We therefore believe that GHR activation by the Ile179Met GH variant could activate JAK 2 normally but not Src, resulting in complete STAT 5 activation but only partial activation of MAPK.

The identification of a GH variant manifesting normal activation of STAT 5 but reduced activation of MAPK in a child exhibiting short stature raises some interesting questions regarding the role that MAPK might play in mediating the action of GH. Previous studies have suggested that the MAPK is not involved in the GH induction of IGF1 gene expression [Shoba et al., Endocrinology 142 3980-3986 (2001); Frago et al, Endocrinology 143 4113-4122 (2002)]. However, it remains possible that this variant may nevertheless still have contributed to the short stature manifested by the proband perhaps while acting in concert with variants at unlinked loci encoding other GH axis proteins. The MAPK pathway may therefore play a greater role than yet identified, in regulating the growth promoting effects of GH.

TABLE 4
GH1 gene lesions found in a screen of 74
individuals with familial short stature

| | Nucleotide | Amino | Confirmed | Proximal |
|---|---|---|---|---|
| | change, | acid | (PCR/ | promoter |
| Patient | location | change | sequencing) | haplotype |
| B4 | −360 A→G | — | Yes | 41 |
| B49 | ATC→ATG, | Ile179Met | Yes | 1 |
| | 1491 | | | |
| B53 | GTC→ATC, | Val110Ile | Yes | 2 or 5? |
| | 1029 | | | |
| B218, | ACA→GCA, | Thr24Ala* | Yes | 21 |
| B401 | 69 | | | |

Nucleotide numbering is based on the GH1 reference sequence (GenBank Accession Number J03071) where +1=transcriptional initiation site. Proximal promoter haplotype numbering after Horan et al. (2003). * Previously reported by Miyata et al. (1997).

1-28. (canceled)

29. An isolated variant of the human growth hormone nucleic acid molecule, GH1, comprising the following substitution: +1491 C→G, wherein 1491 refers to the position of the nucleotide with respect to this transcription initiation site which is designated 1.

30. An isolated variant of the growth hormone nucleic acid molecule, GH1, comprising a nucleic acid molecule that encodes a protein, i.e. a GH protein, including the substitution Ile179Met.

31. An isolated nucleic acid molecule according to claim 29, wherein said molecule is either gDNA, cDNA or mRNA.

32. An isolated nucleic acid molecule according to claim 30, wherein said molecule is either gDNA, cDNA or mRNA.

33. A transcript of the isolated nucleic acid molecule according to claim 29.

34. A transcript of the isolated nucleic acid molecule according to claim 30.

35. A transcript of the isolated nucleic acid molecule according to claim 31.

36. An isolated polypeptide encoded by the nucleic acid molecule according to claim 29.

37. An isolated polypeptide encoded by the isolated nucleic acid molecule according to claim 30.

38. An isolated polypeptide encoded by the isolated nucleic acid molecule according to claim 31.

39. An isolated polypeptide which is a variant of the growth hormone protein, GH, and which includes the substitution Ile179Met.

40. A screening method for screening an individual suspected of having dysfunctional GH which screening method comprises the steps of:
(a) obtaining a test sample comprising a nucleic acid molecule of human GH1 gene from an individual;
(b) sequencing said molecule;
(c) examining said sequence for a +1491C→G substitution; and
(d) where said substitution exists concluding there is a GH dysfunction.

41. A screening method according to claim 40 wherein said sequencing step involves PCR techniques.

42. A screening method for screening an individual suspected of having dysfunctional GH, which screening method comprises the steps of:
(a) obtaining a test sample comprising a growth hormone, GH, polypeptide from said individual;
(b) sequencing said polypeptide;
(c) examining said sequence for a Ile179Met substitution; and
(d) where said substitution exists concluding there is a GH dysfunction.

43. A kit suitable for carrying out the screening method according to claim 40, which kit comprises:
(a) an oligonucleotide having a nucleic acid sequence corresponding to region +1491 of a GH1 gene which region comprises the substitution +1491C→G; and
(b) an oligonucleotide having a nucleic acid sequence corresponding to the wild-type sequence in the region specified in (a); and, optionally,
(c) one or more reagents suitable for carrying out PCR for amplifying desired regions of the patient's DNA.

44. A kit suitable for carrying out the screening method according to claim 42, which kit comprises:
(a) an oligonucleotide having a nucleic acid sequence corresponding to region +1491 of a GH1 gene which region comprises the substitution +1491C→G; and
(b) an oligonucleotide having a nucleic acid sequence corresponding to the wild-type sequence in the region specified in (a); and, optionally,
(c) one or more reagents suitable for carrying out PCR for amplifying desired regions of the patient's DNA.

45. An oligonucleotide suitable for use in the method according to claim 40.

46. An oligonucleotide suitable for use in the method according to claim 42.

47. An oligonucleotide suitable for use in the kit according to claim 43.

48. An oligonucleotide suitable for use in the kit according to claim 44.

49. An isolated growth hormone polypeptide or protein which contains a Ile179Met substitution and which further provides for differential activation of receptor-mediated cell signalling pathways.

50. An isolated polypeptide or protein according to claim 49 wherein said polypeptide or protein activates the STAT5 pathway but shows reduced activation or the MAPK pathway.

51. An isolated polypeptide or protein according to claim 50 wherein said reduction in activity of the MAPK pathway is less than 70% of the activity of the wild-type GH protein.

52. An isolated polypeptide or protein according to claim 51 wherein said reduced activity is less than 50%.

53. An isolated polypeptide or protein according to claim 50 wherein said reduced activity is less than 45%.

54. An isolated growth hormone polypeptide or protein which is characterised by possessing a reduced ability to activate the MAP kinase pathway.

55. An isolated polypeptide or protein according to claim 54 wherein said MAPK pathway is an ERK pathway.

56. A screening method for screening an individual suspected of having dysfunctional GH which screening method comprises the steps of:
(a) obtaining a test sample from said individual comprising the individual's endogenous growth hormone;
(b) examining said growth hormone to determine whether and to what extent it will activate the receptor-mediated MAPK cell signalling pathway; and
(c) where there is a reduction in MAPK cell signalling, with respect to wild-type GH, concluding there is a GH dysfunction.

57. An antibody specific for the isolated growth hormone polypeptide or protein according to claim 49.

58. An antibody specific for the isolated growth hormone polypeptide or protein according to claim 50.

59. An antibody specific for the isolated growth hormone polypeptide or protein according to claim 51.

60. An antibody specific for the isolated growth hormone polypeptide or protein according to claim 52.

61. An antibody specific for the isolated growth hormone polypeptide or protein according to claim 53.

62. An antibody specific for the isolated growth hormone polypeptide or protein according to claim 54.

63. An antibody specific for the isolated growth hormone polypeptide or protein according to claim 55.

64. A pharmaceutical composition comprising a nucleic acid molecule according to claim 29 in association with a pharmaceutically acceptable carrier.

65. A pharmaceutical composition comprising a nucleic acid molecule according to claim 30 in association with a pharmaceutically acceptable carrier.

66. A pharmaceutical composition comprising a nucleic acid molecule according to claim 31 in association with a pharmaceutically acceptable carrier.

67. A pharmaceutical composition comprising a nucleic acid molecule according to claim 32 in association with a pharmaceutically acceptable carrier.

68. A pharmaceutical composition comprising an isolated polypeptide or protein according to claim 49 in association with a pharmaceutically acceptable carrier.

69. A pharmaceutical composition comprising an isolated polypeptide or protein according to claim 50 in association with a pharmaceutically acceptable carrier.

70. A pharmaceutical composition comprising an isolated polypeptide or protein according to claim 51 in association with a pharmaceutically acceptable carrier.

71. A pharmaceutical composition comprising an isolated polypeptide or protein according to claim 52 in association with a pharmaceutically acceptable carrier.

72. A pharmaceutical composition comprising an isolated polypeptide or protein according to claim 53 in association with a pharmaceutically acceptable carrier.

73. A pharmaceutical composition comprising an isolated polypeptide or protein according to claim 54 in association with a pharmaceutically acceptable carrier.

74. A pharmaceutical composition comprising an isolated polypeptide or protein according to claim 55 in association with a pharmaceutically acceptable carrier.

75. A vector comprising a nucleic acid molecule according to claim 29.

76. A vector comprising a nucleic acid molecule according to claim 30.

77. A vector comprising a nucleic acid molecule according to claim 31.

78. A vector comprising a nucleic acid molecule according to claim 32.

79. A host cell comprising a vector according to claim 75.

80. A host cell comprising a vector according to claim 76.

81. A host cell comprising a vector according to claim 77.

82. A host cell comprising a vector according to claim 78.

83. A process for preparing an isolated polypeptide or protein according to claim 49 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1; and
(b) recovering from the culture medium the polypeptide or protein produced by said cell.

84. A process for preparing an isolated polypeptide or protein according to claim 50 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1; and
(b) recovering from the culture medium the polypeptide or protein produced by said cell.

85. A process for preparing an isolated polypeptide or protein according to claim 51 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1; and
(b) recovering from the culture medium the polypeptide or protein produced by said cell.

86. A process for preparing an isolated polypeptide or protein according to claim 52 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1; and
(b) recovering from the culture medium the polypeptide or protein produced by said cell.

87. A process for preparing an isolated polypeptide or protein according to claim 53 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1; and
(b) recovering from the culture medium the polypeptide or protein produced by said cell.

88. A process for preparing an isolated polypeptide or protein according to claim 54 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1; and
(b) recovering from the culture medium the polypeptide or protein produced by said cell.

89. A process for preparing an isolated polypeptide or protein according to claim 55 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1; and
(b) recovering from the culture medium the polypeptide or protein produced by said cell.

90. A process for preparing an isolated polypeptide or protein according to claim 49 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met; and
(b) recovering from the culture medium the polypeptide or protein produced by said cell.

91. A process for preparing an isolated polypeptide or protein according to claim 50 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met; and
(b) recovering from the culture medium the polypeptide or protein produced by said cell.

92. A process for preparing an isolated polypeptide or protein according to claim 51 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met; and
(b) recovering from the culture medium the polypeptide or protein produced by said cell.

93. A process for preparing an isolated polypeptide or protein according to claim 52 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met; and
(b) recovering from the culture medium the polypeptide or protein produced by said cell.

94. A process for preparing an isolated polypeptide or protein according to claim 53 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met; and
(b) recovering from the culture medium the polypeptide or protein produced by said cell.

95. A process for preparing an isolated polypeptide or protein according to claim 54 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met; and
(b) recovering from the culture medium the polypeptide or protein produced by said cell.

96. A process for preparing an isolated polypeptide or protein according to claim 55 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met; and
(b) recovering from the culture medium the polypeptide or protein produced by said cell.

97. A process for preparing an isolated polypeptide or protein according to claim 49 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1, said nucleic acid molecule being either gDNA, cDNA or mRNA; and
(b) recovering from the culture medium the polypeptide or protein produced by said cell.

98. A process for preparing an isolated polypeptide or protein according to claim 50 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1, said nucleic acid molecule being either gDNA, cDNA or mRNA; and
(b) recovering from the culture medium the polypeptide or protein produced by said cell.

99. A process for preparing an isolated polypeptide or protein according to claim 51 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1, said nucleic acid molecule being either gDNA, cDNA or mRNA; and
(b) recovering from the culture medium the polypeptide or protein produced by said cell.

100. A process for preparing an isolated polypeptide or protein according to claim 52 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1, said nucleic acid molecule being either gDNA, cDNA or mRNA; and
(b) recovering from the culture medium the polypeptide or protein produced by said cell.

101. A process for preparing an isolated polypeptide or protein according to claim 53 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1, said nucleic acid molecule being either gDNA, cDNA or mRNA; and
(b) recovering from the culture medium the polypeptide or protein produced by said cell.

102. A process for preparing an isolated polypeptide or protein according to claim 54 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1, said nucleic acid molecule being either gDNA, cDNA or mRNA; and
(b) recovering from the culture medium the polypeptide or protein produced by said cell.

103. A process for preparing an isolated polypeptide or protein according to claim 55 which comprises:
(a) culturing a host cell comprising a vector, said vector comprising a nucleic acid molecule which is an isolated variant of the human growth hormone nucleic acid molecule, GH1, comprising the following substitution: +1491 C→G, wherein 1491 refers to the position of the nucleotide with respect to this transcription initiation site which is designated 1, said nucleic acid molecule being either gDNA, cDNA or mRNA; and
(b) recovering from the culture medium the polypeptide or protein produced by said cell.

104. A process for preparing an isolated polypeptide or protein according to claim 49 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met, said nucleic acid molecule being either gDNA, cDNA or mRNA; and
(b) recovering from the culture medium the polypeptide or protein produced by said cell.

105. A process for preparing an isolated polypeptide or protein according to claim 50 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met, said nucleic acid molecule being either gDNA, cDNA or mRNA; and
(b) recovering from the culture medium the polypeptide or protein produced by said cell.

106. A process for preparing an isolated polypeptide or protein according to claim 51 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met, said nucleic acid molecule being either gDNA, cDNA or mRNA; and
(b) recovering from the culture medium the polypeptide or protein produced by said cell.

107. A process for preparing an isolated polypeptide or protein according to claim 52 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met, said nucleic acid molecule being either gDNA, cDNA or mRNA; and
(b) recovering from the culture medium the polypeptide or protein produced by said cell.

108. A process for preparing an isolated polypeptide or protein according to claim 53 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met, said nucleic acid molecule being either gDNA, cDNA or mRNA; and
(b) recovering from the culture medium the polypeptide or protein produced by said cell.

109. A process for preparing an isolated polypeptide or protein according to claim 54 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met, said nucleic acid molecule being either gDNA, cDNA or mRNA; and
(b) recovering from the culture medium the polypeptide or protein produced by said cell.

110. A process for preparing an isolated polypeptide or protein according to claim 55 which comprises:
(a) culturing a host cell comprising a vector, said vector comprising a nucleic acid molecule which is an isolated variant of the growth hormone nucleic acid molecule, GH1, comprising a nucleic acid molecule that encodes a protein, i.e. a GH protein, including the substitution Ile179Met, said nucleic acid molecule being either gDNA, cDNA or mRNA; and
(b) recovering from the culture medium the polypeptide or protein produced by said cell.

111. A polypeptide or protein produced by the method according to claim 83.

112. A polypeptide or protein produced by the method according to claim 84.

113. A polypeptide or protein produced by the method according to claim 85.

114. A polypeptide or protein produced by the method according to claim 86.

115. A polypeptide or protein produced by the method according to claim 87.

116. A polypeptide or protein produced by the method according to claim 88.

117. A polypeptide or protein produced by the method according to claim 89.

118. A polypeptide or protein produced by the method according to claim 90.

119. A polypeptide or protein produced by the method according to claim 91.

120. A polypeptide or protein produced by the method according to claim 92.

121. A polypeptide or protein produced by the method according to claim 93.

122. A polypeptide or protein produced by the method according to claim 94.

123. A polypeptide or protein produced by the method according to claim 95.

124. A polypeptide or protein produced by the method according to claim 96.

125. A polypeptide or protein produced by the method according to claim 97.

126. A polypeptide or protein produced by the method according to claim 98.

127. A polypeptide or protein produced by the method according to claim 99.

128. A polypeptide or protein produced by the method according to claim 100.

129. A polypeptide or protein produced by the method according to claim 101.

130. A polypeptide or protein produced by the method according to claim 102.

131. A polypeptide or protein produced by the method according to claim 103.

132. A polypeptide or protein produced by the method according to claim 104.

133. A polypeptide or protein produced by the method according to claim 105.

134. A polypeptide or protein produced by the method according to claim 106.

135. A polypeptide or protein produced by the method according to claim 107.

136. A polypeptide or protein produced by the method according to claim 108.

137. A polypeptide or protein produced by the method according to claim 109.

138. A polypeptide or protein produced by the method according to claim 110.

139. A transcript of the isolated nucleic acid molecule according to claim 32.

140. An isolated polypeptide encoded by the isolated nucleic acid molecule according to claim 32.


SEQUENCE LISTING (2 sequences)

SEQ ID NO: 1 - DNA, 3700 bp, human
ctgtttcttggtttgtgtctctgctgcaagtccaaggagctggggcaataccttgagtctgggttcttcgtccccagggacctgggggagccccagcaatgctcagggaaaggggagagcaaagtgtggggttggttctctctagtggtcagtgttggaactgcatccagctgactcaggctgacccaggagtcctcagcagaagtggaattcaggactgaatcgtgctcacaacccccacaatctattggctgtgcttggccccttttcccaacacacacattctgtctggtgggtggaggttaaacatgcggggaggaggaaagggataggatagagaatgggatgtggtcggtagggggtctcaaggactggctatcctgacatccttctccgcgttcaggttggccaccatggcctgcggccagagggcacccacgtgacccttaaagagaggacaagttgggtggtatctctggctgacactctgtgcacaaccctcacaacactggtgacggtgggaagggaaagatgacaagccagggggcatgatcccagcatgtgtgggaggagcttctaaattatccattagcacaagcccgtcagtggccccatgcataaatgtacacagaaacaggtgggggcaacagtgggagagaaggggccagggtataaaaagggcccacaagagaccagctcaaggatcccaaggcccaactccccgaaccactcagggtcctgtggacagctcacctagcggcaatggctacaggtaagcgcccctaaaatccctttgggcacaatgtgtcctgaggggagaggcagcgacctgtagatgggacgggggcactaaccctcaggtttggggcttctgaatgtgagtatcgccatgtaagcccagtatttggccaatctcagaaagctcctggtccctggagggatggagagagaaaaacaaacagctcctggagcagggagagtgctggcctcttgctctccggctccctctgttgccctctggtttctccccaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgtaagctcttggggaatgggtgcgcatcaggggtggcaggaaggggtgactttcccccgctgggaaataagaggaggagactaaggagctcagggtttttcccgaagcgaaaatgcaggcagatgagcacacgctgagtgaggttcccagaaaagtaacaatgggagctggtctccagcgtagaccttggtgggcggtccttctcctaggaagaagcctatatcccaaaggaacagaagtattcattcctgcagaacccccagacctccctctgtttctcagagtctattccgacaccctccaacagggaggaaacacaacagaaatccgtgagtggatgccttctccccaggcggggatgggggagacctgtagtcagagcccccgggcagcacagccaatgcccgtccttcccctgcagaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatgggggtgagggtggcgccaggggtccccaatcctggagccccactgactttgagagctgtgttagagaaacactgctgccctctttttagcagtcaggccctgacccaagagaactcaccttattcttcatttcccctcgtgaatcctccaggcctttctctacaccctgaaggggagggaggaaaatgaatgaatgagaaagggagggaacagtacccaagcgcttggcctctccttctcttccttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattcgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctgccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagaacttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatggatcgtctccctggagaataatccccaaagtgaaattacttagcacgtccagttaggtagatccttgtgtacttcttggttgttcagagatcatcaaccagtgcaaacaatccccccatcaatacacagcagtgcctgcccctctccccccgaggtcttccgaggcccttcctccgtgcctgaaccccctggacatatcatatggcaaactgaagtgtccaacgagatataggaagtgaaacacgatgtacactgaaacgtgcaatacaaatatgcagcatgaagtgcctcggttcactaacccgagctacgctgggtgcttcttttctaccactttcctta

SEQ ID NO: 2 - protein, 191 aa, human [sequence omitted]